(19) United States
(12) Patent Application Publication
(10) Pub. No.: US 2014/0271908 A1
(43) Pub. Date: Sep. 18, 2014

(54) DIETARY SUPPLEMENTS AND METHODS FOR PREVENTING AND TREATING MIGRAINES

(71) Applicant: Melaleuca, Inc., Idaho Falls, ID (US)

(72) Inventor: Jeremy Ivie, Ammon, ID (US)

(73) Assignee: Melaleuca, Inc., Idaho Falls, ID (US)

(21) Appl. No.: 14/212,093

(22) Filed: Mar. 14, 2014

Related U.S. Application Data
(60) Provisional application No. 61/794,918, filed on Mar. 15, 2013.

(51) Int. Cl. A61K 31/353; A61K 31/122; A61K 31/685; A61K 31/525; A61K 36/15; A61K 35/64; A61K 35/60; A61K 31/702

(52) U.S. Cl. CPC A61K 31/353; A61K 35/60; A61K 31/122; A61K 31/702; A61K 31/525; A61K 36/15; A61K 35/644; A61K 31/685
USPC 424/523

(57) ABSTRACT

This document provides methods and materials related to preventing and treating migraines. For example, compositions (e.g., dietary supplements) containing one or more of coenzyme Q10, magnesium, riboflavin, pine bark extract, and fish oil are provided.

### CROSS-REFERENCE TO RELATED APPLICATIONS

This application claims priority to U.S. Provisional Application Ser. No. 61/794,918, filed on Mar. 15, 2013, which is herein incorporated by reference in its entirety.

### BACKGROUND

1. Technical Field

This document relates to dietary supplements as well as methods and materials for preventing and treating migraines.

2. Background Information

A migraine is a chronic, episodic, and debilitating primary headache syndrome that affects about 15 to 20% of the world population. Arunagiri et al., Curr. Opin. Opthalmol 14:344-352 (2003). Migraines are generally classified as one of two main types. The first type, migraine without aura (previously known as common migraine), affects about 15% of the population. In migraine without aura, the headache is usually unilateral, pulsating, and moderate to severe in intensity, and may last a few hours to 3 days. The headache may also be associated with nausea, vomiting, photophobia, phonophobia, and other symptoms. The second type, migraine with aura (previously known as classic migraine), affects about 8% of the population. In migraine with aura, one or more auras, such as visual, somatosensory, and motor symptoms, develop prior to the development of a migraine attack. Migraine without aura and migraine with aura co-occur in 13% of migraineurs.

### SUMMARY

This document provides methods and materials related to the prevention and treatment of migraines. For example, the document provides compositions (e.g., dietary supplements) containing one or more of coenzyme Q10, magnesium, riboflavin, pine bark extract, and fish oil.

In some cases, provided herein is a dietary supplement comprising Coenzyme Q10, magnesium, and riboflavin, wherein the improvement comprises: pine bark extract and fish oil in combination with the Coenzyme Q10, magnesium, and riboflavin.

In some cases, the supplement can further comprise one or more of beeswax and lecithin oil.

In some cases, the magnesium can be a magnesium salt or a complex containing magnesium. For example, a complex containing magnesium can be a magnesium amino acid polysaccharide complex.

In some cases, a dietary supplement as provided herein can include:

from about 1% to about 4% by weight Coenzyme Q10;
from about 20% to about 60% by weight magnesium;
from about 20% to about 60% by weight riboflavin;
from about 0.25% to about 2% by weight pine bark extract; and
from about 20% to about 60% by weight fish oil.

For example, a dietary supplement as provided herein can include:

from about 2.75% to about 3.25% by weight Coenzyme Q10;
from about 42.5% to about 47.5% by weight magnesium;
from about 42.5% to about 47.5% by weight riboflavin;
from about 0.8% to about 1.2% by weight pine bark extract; and
from about 38% to about 42% by weight fish oil.

In some cases, a dietary supplement as provided herein can include:

from about 15 mg to about 45 mg of Coenzyme Q10;
from about 50 mg to about 150 mg magnesium;
from about 50 mg to about 150 mg riboflavin;
from about 6 mg to about 18 mg pine bark extract; and
from about 200 mg to about 800 mg fish oil.

For example, a dietary supplement as provided herein can include:

about 37.5 mg of Coenzyme Q10;
about 100 mg magnesium;
about 100 mg riboflavin;
about 12.5 mg pine bark extract; and
about 550 mg fish oil.

Further provided herein is a method for preventing or treating a migraine in a mammal. In some cases, the method can include administering to the mammal a therapeutically effective amount of a dietary supplement provided herein. For example, a method for reducing one or more symptoms associated with a migraine in a mammal is provided, where the method can include administering to the mammal a therapeutically effective amount of a dietary supplement provided herein. In some cases, the administering occurs at least once daily for at least 28 consecutive days.

In some cases, the one or more symptoms associated with a migraine are selected from the group consisting of: the frequency of headache, the severity of headache, the duration of headache, and the degree of migraine disability. In some cases, the one or more symptoms associated with a migraine are selected from the group consisting of: headache, nausea, vomiting, photophobia, phonophobia, and aura.

In some cases, a daily dosage of the dietary supplement can include:

from about 100 mg to about 300 mg Coenzyme Q10;
from about 200 mg to about 600 mg magnesium;
from about 200 mg to about 600 mg riboflavin;
from about 20 mg to about 80 mg pine bark extract; and
from about 1000 mg to about 3000 mg fish oil.

Also provided herein is a method for preventing or treating a migraine in a mammal. In some cases, the method can include administering to the mammal a therapeutically effective amount of a dietary supplement provided herein, wherein the administering is at least once daily for at least 28 consecutive days (e.g., at least 56 days, at least 84 days, and at least 112 days).

Unless defined otherwise, all technical and scientific terms used herein have the same meaning as is commonly understood by one of ordinary skill in the art to which this disclosure pertains. All patents, applications, published applications, and other publications are incorporated by reference in their entirety. In the event that there is a plurality of definitions for a term herein, those in this section prevail unless stated otherwise.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a line graph comparing headache frequency between Group 1 (test group) and Group 2 (control) over a 112 day period.

FIG. 2 is a line graph showing the percentage of individuals that had a greater than 40% improvement in headache frequency at various time points.

FIG. 3 is a line graph comparing headache severity between Group 1 (test group) and Group 2 (control) over a 112 day period.

FIG. 4 is a line graph showing the percentage of individuals that had a greater than 40% improvement in headache severity at various time points.

FIG. 5 is a line graph comparing headache duration between Group 1 (test group) and Group 2 (control) over a 112 day period.

FIG. 6 is a line graph showing the percentage of individuals that had a greater than 40% improvement in headache duration at various time points.

### DETAILED DESCRIPTION

This document provides methods and materials related to treating and preventing one or more symptoms of a migraine. For example, the compositions provided herein can be used to reduce the frequency of headache, the severity of headache, the duration of headache, and/or the degree of migraine disability. In some cases, compositions provided herein (e.g., dietary supplements) can contain one or more of Coenzyme Q10, magnesium, riboflavin, pine bark extract, and fish oil.

### Coenzyme Q10

A composition provided herein can contain Coenzyme Q10 (CoQ10; ubiquinone, ubidecarenone, and coenzyme Q). CoQ10 can be synthesized or isolated from natural sources. In some cases, CoQ10 can be obtained commercially.

In some cases, a composition provided herein can contain between about 0.5% and 10% by weight of CoQ10. For example, about 1% to about 10% by weight, about 1.5% to about 10% by weight, about 2% to about 10% by weight, about 2.2% to about 10% by weight, about 2.5% to about 10% by weight, about 2.75% to about 10% by weight, about 3% to about 10% by weight, about 3.1% to about 10% by weight, about 3.5% to about 10% by weight, about 4% to about 10% by weight, about 4.4% to about 10% by weight; about 5% to about 10% by weight, about 5.25% to about 10% by weight, about 6% to about 10% by weight, about 6.4% to about 10% by weight, about 6.8% to about 10% by weight, about 7% to about 10% by weight, about 7.5% to about 10% by weight, about 0.5% to about 9% by weight, about 0.5% to about 8.5% by weight, about 0.5% to about 7% by weight, about 0.5% to about 6.5% by weight, about 0.5% to about 6% by weight, about 0.5% to about 5.4% by weight, about 0.5% to about 5% by weight, about 0.5% to about 4.6% by weight, about 0.5% to about 4% by weight, about 0.5% to about 3.5% by weight, about 0.5% to about 3.1% by weight, about 0.5% to about 3% by weight, about 0.5% to about 2.5% by weight, about 0.5% to about 2% by weight, about 0.5% to about 1.6% by weight, 0.5% to about 1% by weight, about 1% to about 4% by weight, about 1.5% to about 3.5% by weight, about 2% to about 4% by weight, about 2.5% to about 3.5% by weight, or about 2.75% to about 3.25% by weight of a dietary supplement can be CoQ10. In some embodiments, a composition provided herein can contain between about 1% to about 4% by weight of CoQ10 (e.g., about 1.5% to about 3.5% by weight, about 2% to about 4% by weight, about 2.5% to about 3.5% by weight, or about 2.75% to about 3.25% by weight). For example, a composition provided herein can contain about 3.02% by weight CoQ10.

Typically, a dietary supplement provided herein can contain between about 5 mg to about 75 mg of CoQ10 (e.g., about 5 mg to about 70 mg, about 5 mg to about 65 mg, about 5 mg to about 60 mg, about 5 mg to about 55 mg, about 5 mg to about 50 mg, about 5 mg to about 47.5 mg, about 5 mg to about 45 mg, about 5 mg to about 42.5 mg, about 5 mg to about 40 mg, about 5 mg to about 38 mg, about 5 mg to about 37.5 mg, about 5 mg to about 35 mg, about 5 mg to about 30 mg, about 5 mg to about 25 mg, about 5 mg to about 20 mg, about 5 mg to about 15 mg, about 10 mg to about 75 mg, about 15 mg to about 75 mg, about 20 mg to about 75 mg, about 25 mg to about 75 mg, about 30 mg to about 75 mg, about 32.5 mg to about 75 mg, about 35 mg to about 75 mg, about 37.5 mg to about 75 mg, about 40 mg to about 75 mg, about 45 mg to about 75 mg, about 50 mg to about 75 mg, about 55 mg to about 75 mg, about 60 mg to about 75 mg, about 15 mg to about 45 mg, about 20 mg to about 40 mg, about 30 mg to about 40 mg, about 35 mg to about 40 mg, and about 32.5 mg to about 42.5 mg). In some embodiments, a dietary supplement can contain between about 15 mg to about 45 mg of CoQ10 (e.g., about 20 mg to about 40 mg, about 30 mg to about 40 mg, about 35 mg to about 40 mg, and about 32.5 mg to about 42.5 mg). For example, a composition provided herein can contain about 37.5 mg of CoQ10.

In some cases, a composition provided herein can be formulated to contain an amount of CoQ10 such that a daily dose of between about 50 mg and about 500 mg (e.g., between about 50 and about 400 mg, between about 50 mg and about 300 mg, between about 50 mg and about 250 mg, between about 50 mg and about 200 mg, between about 50 mg and about 150 mg, between about 100 mg and about 500 mg, between about 125 mg and about 500 mg, between about 150 mg and about 500 mg, between about 100 mg and about 300 mg, between about 125 mg and about 175 mg, between about 100 mg and about 200 mg, and between about 75 mg and about 225 mg) of CoQ10 is administered. In some embodiments, a composition provided herein can be formulated to contain an amount of CoQ10 such that a daily dose of between about 100 mg and about 300 mg (e.g., between about 125 mg and about 175 mg, between about 100 mg and about 200 mg, and between about 75 mg and about 225 mg) is administered. For example, a composition can be formulated such that a daily dose of about 150 mg of CoQ10 is administered.

### Magnesium

A composition provided herein can include magnesium. Any biologically useful form of magnesium can be used. For example, magnesium sulfate, magnesium hydroxide, magnesium chloride, magnesium oxide, magnesium gluconate, magnesium malate, magnesium orotate, magnesium glycinate, magnesium citrate, and magnesium lactate can be used. In some cases, the magnesium can be in the form of a magnesium amino acid polysaccharide complex (also known as Magnesium Oligo) as described in U.S. Pat. No. 8,273,393, which is incorporated by reference in its entirety. In some cases, magnesium or a salt or complex containing magnesium can be synthesized or obtained commercially.

In some cases, a composition provided herein can contain between about 15% and 75% by weight of a salt or complex containing magnesium. For example, about 20% to about 75% by weight, about 25% to about 75% by weight, about 32.5% to about 75% by weight, about 35% to about 75% by weight, about 37.5% to about 75% by weight, about 40% to about 75% by weight, about 42.5% to about 75% by weight, about 45% to about 75% by weight, about 50% to about 75% by weight, about 55% to about 75% by weight, about 15% to about 70% by weight; about 15% to about 65% by weight, about 15% to about 60% by weight, about 15% to about 55% by weight, about 15% to about 50% by weight, about 15% to about 47.5% by weight, about 15% to about 45% by weight, about 15% to about 42.5% by weight, about 20% to about 60% by weight, about 25% to about 50% by weight, about 30% to about 50% by weight, about 35% to about 50% by weight, about 40% to about 45% by weight, about 37.5% to about 47.5% by weight, and about 42.5% to about 47.5% by weight of a dietary supplement can be a salt or complex containing magnesium. In some embodiments, a composition provided herein can be formulated to contain an amount of a salt or complex containing magnesium of between about 20% to about 60% by weight (e.g., about 25% to about 50% by weight, about 30% to about 50% by weight, about 35% to about 50% by weight, about 40% to about 45% by weight, about 37.5% to about 47.5% by weight, and about 42.5% to about 47.5% by weight). For example, a composition provided herein can contain about 43.3% by weight of a salt or complex containing magnesium.

Typically, a dietary supplement provided herein can contain between about 25 mg to about 250 mg of magnesium (e.g., about 25 mg to about 200 mg, about 25 mg to about 175 mg, about 25 mg to about 150 mg, about 25 mg to about 120 mg, about 25 mg to about 115 mg, about 25 mg to about 110 mg, about 25 mg to about 100 mg, about 25 mg to about 75 mg, about 25 mg to about 50 mg, about 50 mg to about 250 mg, about 75 mg to about 250 mg, about 80 mg to about 250 mg, about 85 mg to about 250 mg, about 90 mg to about 250 mg, about 95 mg to about 250 mg, about 100 mg to about 250 mg, about 125 mg to about 250 mg, about 150 mg to about 250 mg, about 200 mg to about 250 mg, about 50 mg to about 150 mg, about 75 mg to about 125 mg, about 80 mg to about 120 mg, about 90 mg to about 110 mg, about 95 mg to about 105 mg). In some embodiments, a dietary supplement provided herein can contain between about 50 mg to about 150 mg of magnesium (e.g., about 75 mg to about 125 mg, about 80 mg to about 120 mg, about 90 mg to about 110 mg, about 95 mg to about 105 mg). For example, a dietary supplement provided herein can contain about 100 mg of a salt or complex containing magnesium. In some embodiments, the magnesium may be present as part of a salt or complex containing the magnesium. The salt or complex containing the magnesium will have a higher weight as compared to the magnesium alone.

A composition provided herein can be formulated to contain an amount of a salt or complex containing magnesium such that a daily dose of magnesium between about 100 mg and about 1200 mg (e.g., between about 100 and about 1000 mg, between about 100 mg and about 800 mg, between about 100 mg and about 600 mg, between about 100 mg and about 500 mg, between about 100 mg and about 450 mg, between about 200 mg and about 1200 mg, between about 250 mg and about 1200 mg, between about 300 mg and about 1200 mg, between about 350 mg and about 1200 mg, between about 600 mg and about 1200 mg, between about 800 mg and about 1200 mg, between about 200 mg and about 600 mg, between about 250 mg and about 550 mg, between about 300 mg and about 500 mg, between about 350 mg and about 450 mg, and between about 375 mg and about 425 mg) is administered. In some embodiments, a composition provided herein can be formulated to contain an amount of a salt or complex containing magnesium such that a daily dose of magnesium between about 200 mg and about 600 mg (e.g., between about 250 mg and about 550 mg, between about 300 mg and about 500 mg, between about 350 mg and about 450 mg, and between about 375 mg and about 425 mg) is administered. For example, a composition provided herein can be formulated such that a daily dose of about 400 mg of magnesium is administered.

In some cases, the salt or complex containing magnesium can be a magnesium amino acid polysaccharide complex (also known as Magnesium Oligo). In such cases, a composition provided herein can contain between about 15% and 75% by weight of a magnesium amino acid polysaccharide complex. For example, about 20% to about 75% by weight, about 25% to about 75% by weight, about 32.5% to about 75% by weight, about 35% to about 75% by weight, about 37.5% to about 75% by weight, about 40% to about 75% by weight, about 42.5% to about 75% by weight, about 45% to about 75% by weight, about 50% to about 75% by weight, about 55% to about 75% by weight, about 15% to about 70% by weight; about 15% to about 65% by weight, about 15% to about 60% by weight, about 15% to about 55% by weight, about 15% to about 50% by weight, about 15% to about 47.5% by weight, about 15% to about 45% by weight, about 15% to about 42.5% by weight, about 20% to about 60% by weight, about 25% to about 50% by weight, about 30% to about 50% by weight, about 35% to about 50% by weight, about 40% to about 45% by weight, about 37.5% to about 47.5% by weight, and about 42.5% to about 47.5% by weight of a dietary supplement provided herein can be a magnesium amino acid polysaccharide complex. In some embodiments, a composition provided herein can be formulated to contain an amount of a magnesium amino acid polysaccharide complex of between about 20% to about 60% by weight (e.g., about 25% to about 50% by weight, about 30% to about 50% by weight, about 35% to about 50% by weight, about 40% to about 45% by weight, about 37.5% to about 47.5% by weight, and about 42.5% to about 47.5% by weight) is administered. For example, a composition provided herein can contain about 43.3% of a magnesium amino acid polysaccharide complex.

Typically, a dietary supplement provided herein can contain between about 25 mg to about 250 mg of magnesium as a component of a magnesium amino acid polysaccharide complex (e.g., about 25 mg to about 200 mg, about 25 mg to about 175 mg, about 25 mg to about 150 mg, about 25 mg to about 120 mg, about 25 mg to about 115 mg, about 25 mg to about 110 mg, about 25 mg to about 100 mg, about 25 mg to about 75 mg, about 25 mg to about 50 mg, about 50 mg to about 250 mg, about 75 mg to about 250 mg, about 80 mg to about 250 mg, about 85 mg to about 250 mg, about 90 mg to about 250 mg, about 95 mg to about 250 mg, about 100 mg to about 250 mg, about 125 mg to about 250 mg, about 150 mg to about 250 mg, about 200 mg to about 250 mg, about 50 mg to about 150 mg, about 75 mg to about 125 mg, about 80 mg to about 120 mg, about 90 mg to about 110 mg, about 95 mg to about 105 mg). In some embodiments, a dietary supplement provided herein can contain between about 50 mg to about 150 mg of magnesium as a component of a magnesium amino acid polysaccharide complex (e.g., about 75 mg to about 125 mg, about 80 mg to about 120 mg, about 90 mg to about 110 mg, about 95 mg to about 105 mg). For example, a dietary supplement provided herein can contain about 100 mg of magnesium as a component of a magnesium amino acid polysaccharide complex.

A composition provided herein can be formulated to contain an amount of magnesium as a component of a magnesium amino acid polysaccharide complex such that a daily dose of between about 100 mg and about 1200 mg (e.g., between about 100 and about 1000 mg, between about 100 mg and about 800 mg, between about 100 mg and about 600 mg, between about 100 mg and about 500 mg, between about 100 mg and about 450 mg, between about 200 mg and about 1200 mg, between about 250 mg and about 1200 mg, between about 300 mg and about 1200 mg, between about 350 mg and about 1200 mg, between about 600 mg and about 1200 mg, between about 800 mg and about 1200 mg, between about 200 mg and about 600 mg, between about 250 mg and about 550 mg, between about 300 mg and about 500 mg, between about 350 mg and about 450 mg, and between about 375 mg and about 425 mg) of magnesium is administered. In some embodiments, a composition provided herein can be formulated to contain an amount of magnesium as a component of a magnesium amino acid polysaccharide complex such that a daily dose of between about 200 mg and about 600 mg (e.g., between about 250 mg and about 550 mg, between about 300 mg and about 500 mg, between about 350 mg and about 450 mg, and between about 375 mg and about 425 mg) of magnesium is administered. For example, a composition provided herein can be formulated such that a daily dose of about 400 mg of magnesium as a component of a magnesium amino acid polysaccharide complex is administered.

### Riboflavin (Vitamin B2)

A composition provided herein can include riboflavin, also known as Vitamin B2. In some cases, riboflavin can be obtained commercially.

In some cases, a composition provided herein can contain between about 1% and 20% by weight of riboflavin. For example, about 2% to about 20% by weight, about 4% to about 20% by weight, about 5% to about 20% by weight, about 6% to about 20% by weight, about 6.5% to about 20% by weight, about 7% to about 20% by weight, about 7.5% to about 20% by weight, about 8% to about 20% by weight, about 10% to about 20% by weight, about 12% to about 20% by weight, about 15% to about 20% by weight; about 1% to about 15% by weight, about 1% to about 12% by weight, about 1% to about 10% by weight, about 1% to about 9% by weight, about 1% to about 8.5% by weight, about 1% to about 8% by weight, about 1% to about 6% by weight, about 1% to about 4% by weight, about 2% to about 14% by weight, about 3% to about 13% by weight, about 4% to about 12% by weight, about 5% to about 11% by weight, about 6% to about 10% by weight, about 7.5% to about 9.5% by weight, and about 8% to about 9% by weight of a dietary supplement provided herein can be riboflavin. In some embodiments, a composition provided herein can be formulated to contain an amount of riboflavin of between about 2% to about 14% by weight (e.g., about 3% to about 13% by weight, about 4% to about 12% by weight, about 5% to about 11% by weight, about 6% to about 10% by weight, about 7.5% to about 9.5% by weight, and about 8% to about 9% by weight). For example, a composition provided herein can contain about 8.67% by weight of riboflavin.

Typically, a dietary supplement provided herein can contain between about 25 mg to about 250 mg of riboflavin (e.g., about 25 mg to about 200 mg, about 25 mg to about 175 mg, about 25 mg to about 150 mg, about 25 mg to about 120 mg, about 25 mg to about 115 mg, about 25 mg to about 110 mg, about 25 mg to about 100 mg, about 25 mg to about 75 mg, about 25 mg to about 50 mg, about 50 mg to about 250 mg, about 75 mg to about 250 mg, about 80 mg to about 250 mg, about 85 mg to about 250 mg, about 90 mg to about 250 mg, about 95 mg to about 250 mg, about 100 mg to about 250 mg, about 125 mg to about 250 mg, about 150 mg to about 250 mg, about 200 mg to about 250 mg, about 50 mg to about 150 mg, about 75 mg to about 125 mg, about 80 mg to about 120 mg, about 90 mg to about 110 mg, about 95 mg to about 105 mg). In some embodiments, a dietary supplement provided herein can contain between about 50 mg to about 150 mg of riboflavin (e.g., about 75 mg to about 125 mg, about 80 mg to about 120 mg, about 90 mg to about 110 mg, about 95 mg to about 105 mg). For example, a dietary supplement provided herein can contain about 100 mg of riboflavin.

A composition provided herein can be formulated to contain an amount of riboflavin such that a daily dose of between about 100 mg and about 1200 mg (e.g., between about 100 and about 1000 mg, between about 100 mg and about 800 mg, between about 100 mg and about 600 mg, between about 100 mg and about 500 mg, between about 100 mg and about 450 mg, between about 200 mg and about 1200 mg, between about 250 mg and about 1200 mg, between about 300 mg and about 1200 mg, between about 350 mg and about 1200 mg, between about 600 mg and about 1200 mg, between about 800 mg and about 1200 mg, between about 200 mg and about 600 mg, between about 250 mg and about 550 mg, between about 300 mg and about 500 mg, between about 350 mg and about 450 mg, and between about 375 mg and about 425 mg) of riboflavin is administered. In some embodiments, a composition provided herein can be formulated to contain an amount of riboflavin such that a daily dose of between about 200 mg and about 600 mg (e.g., between about 250 mg and about 550 mg, between about 300 mg and about 500 mg, between about 350 mg and about 450 mg, and between about 375 mg and about 425 mg) is administered. For example, a composition provided herein can be formulated such that a daily dose of about 100 mg riboflavin is administered.

### Pine Bark Extract

A composition provided herein can include pine bark extract. Pine bark extract is also known as pygenol, Pycnogenol, and Masquelier's Original OPCs (oligomeric proanthocyanidins). In some embodiments, pine bark extract is made from the bark of the maritime pine tree (Pinus pinaster), which contains naturally occurring chemicals called proanthocyanidins. An extract of pine bark can be prepared using a water extraction. In some cases, pine bark extract can be obtained commercially.

In some cases, a composition provided herein can contain between about 0.1% and 5% by weight of pine bark extract. For example, about 0.1% to about 4% by weight, about 0.1% to about 3% by weight, about 0.1% to about 2.5% by weight, about 0.1% to about 2% by weight, about 0.1% to about 1.75% by weight, about 0.1% to about 1.5% by weight, about 0.1% to about 1.25% by weight, about 0.1% to about 1% by weight, about 0.1% to about 0.75% by weight, about 0.1% to about 0.5% by weight, about 0.25% to about 5% by weight; about 0.5% to about 5% by weight, about 0.75% to about 5% by weight, about 1% to about 5% by weight, about 1.25% to about 5% by weight, about 1.5% to about 5% by weight, about 2% to about 5% by weight, about 3% to about 5% by weight, about 0.25% to about 2% by weight, about 0.5% to about 1.5% by weight, about 0.75% to about 1.25% by weight, about 0.8% to about 1.2% by weight of a dietary supplement can be pine bark extract. In some embodiments, a composition provided herein can be formulated to contain an amount of pine bark extract of between about 0.25% to about 2% by weight (e.g., about 0.5% to about 1.5% by weight, about 0.75% to about 1.25% by weight, about 0.8% to about 1.2% by weight). For example, a composition provided herein can contain about 1.06% by weight pine bark extract.

Typically, a dietary supplement provided herein can contain between about 4 mg to about 40 mg of proanthocyanidins from pine bark extract (e.g., about 4 mg to about 35 mg, about 4 mg to about 30 mg, about 4 mg to about 25 mg, about 4 mg to about 20 mg, about 4 mg to about 15 mg, about 4 mg to about 13 mg, about 4 mg to about 10 mg, about 4 mg to about 8 mg, about 6 mg to about 40 mg, about 8 mg to about 40 mg, about 10 mg to about 40 mg, about 11 mg to about 40 mg, about 12 mg to about 40 mg, about 15 mg to about 40 mg, about 20 mg to about 40 mg, about 25 mg to about 40 mg, about 30 mg to about 40 mg, about 6 mg to about 18 mg, about 8 mg to about 16 mg, about 10 mg to about 14 mg, about 11 mg to about 13 mg, and about 12 mg to about 13 mg). In some embodiments, a dietary supplement provided herein can contain between about 6 mg to about 18 mg of proanthocyanidins from pine bark extract (e.g., about 8 mg to about 16 mg, about 10 mg to about 14 mg, about 11 mg to about 13 mg, and about 12 mg to about 13 mg). For example, a dietary supplement provided herein can contain about 12.5 mg of proanthocyanidins from pine bark extract.

A composition provided herein can be formulated to contain an amount of proanthocyanidins from pine bark extract such that a daily dose of between about 10 mg and about 200 mg (e.g., between about 10 and about 175 mg, between about 10 mg and about 150 mg, between about 10 mg and about 125 mg, between about 10 mg and about 100 mg, between about 10 mg and about 75 mg, between about 10 mg and about 70 mg, between about 10 mg and about 65 mg, between about 10 mg and about 60 mg, between about 10 mg and about 50 mg, between about 10 mg and about 40 mg, between about 10 mg and about 30 mg, between about 20 mg and about 200 mg, between about 30 mg and about 200 mg, between about 40 mg and about 200 mg, between about 45 mg and about 200 mg, between about 50 mg and about 200 mg, between about 75 mg and about 200 mg, between about 100 mg and about 200 mg, between about 150 mg and about 200 mg, between about 20 mg and about 80 mg, between about 25 mg and about 75 mg, between about 30 mg and about 70 mg, between about 40 mg and about 60 mg, and between about 45 mg and about 55 mg) of proanthocyanidins are administered. In some embodiments, a composition provided herein can be formulated to contain an amount of pine bark extract such that a daily dose of between about 20 mg and about 80 mg (e.g., between about 25 mg and about 75 mg, between about 30 mg and about 70 mg, between about 40 mg and about 60 mg, and between about 45 mg and about 55 mg) of proanthocyanidins are administered. For example, a composition provided herein can be formulated such that a daily dose of about 50 mg of proanthocyanidins are administered.

### Fish Oil

A composition provided herein can include fish oil. Fish oil can come in an ethyl ester (EE) or triglyceride (TG) form. In some embodiments, a composition provided herein includes the TG form of fish oil (e.g., Fish Oil TG 18/2). In some cases, fish oil can be obtained commercially.

In some cases, a composition provided herein can contain between about 15% and 75% by weight of fish oil. For example, about 20% to about 75% by weight, about 25% to about 75% by weight, about 32.5% to about 75% by weight, about 35% to about 75% by weight, about 37.5% to about 75% by weight, about 40% to about 75% by weight, about 42.5% to about 75% by weight, about 45% to about 75% by weight, about 50% to about 75% by weight, about 55% to about 75% by weight, about 15% to about 70% by weight; about 15% to about 65% by weight, about 15% to about 60% by weight, about 15% to about 55% by weight, about 15% to about 50% by weight, about 15% to about 47.5% by weight, about 15% to about 45% by weight, about 15% to about 42.5% by weight, about 20% to about 60% by weight, about 25% to about 50% by weight, about 30% to about 50% by weight, about 35% to about 45% by weight, about 37.5% to about 42.5% by weight, and about 38% to about 42% by weight of a dietary supplement can be fish oil. In some embodiments, a composition provided herein can be formulated to contain an amount of fish oil of between about 20% to about 60% by weight (e.g., about 25% to about 50% by weight, about 30% to about 50% by weight, about 35% to about 45% by weight, about 37.5% to about 42.5% by weight, and about 38% to about 42% by weight). For example, a composition provided herein can be formulated to contain about 40.6% by weight of fish oil.

Typically, a dietary supplement provided herein can contain between about 150 mg to about 1000 mg of fish oil (e.g., about 150 mg to about 900 mg, about 150 mg to about 800 mg, about 150 mg to about 750 mg, about 150 mg to about 700 mg, about 150 mg to about 650 mg, about 150 mg to about 600 mg, about 150 mg to about 550 mg, about 150 mg to about 500 mg, about 150 mg to about 450 mg, about 150 mg to about 400 mg, about 150 mg to about 300 mg, about 200 mg to about 1000 mg, about 250 mg to about 1000 mg, about 300 mg to about 1000 mg, about 350 mg to about 1000 mg, about 400 mg to about 1000 mg, about 450 mg to about 1000 mg, about 500 mg to about 1000 mg, about 550 mg to about 1000 mg, about 600 mg to about 1000 mg, about 650 mg to about 1000 mg, about 700 mg to about 1000 mg, about 500 mg to about 1000 mg, about 200 mg to about 800 mg, about 250 mg to about 750 mg, about 300 mg to about 650 mg, about 350 mg to about 600 mg, about 450 mg to about 650 mg, about 500 mg to about 600 mg, and about 525 mg to about 575 mg). In some embodiments, a dietary supplement provided herein can contain between about 200 mg to about 800 mg of fish oil (e.g., about 250 mg to about 750 mg, about 300 mg to about 650 mg, about 350 mg to about 600 mg, about 450 mg to about 650 mg, about 500 mg to about 600 mg, and about 525 mg to about 575 mg). For example, a dietary supplement provided herein can contain about 550 mg of fish oil.

A composition provided herein can be formulated to contain an amount of fish oil such that a daily dose of between about 500 mg and about 5000 mg (e.g., between about 500 and about 4000 mg, between about 500 mg and about 3500 mg, between about 500 mg and about 3000 mg, between about 500 mg and about 2750 mg, between about 500 mg and about 2500 mg, between about 500 mg and about 2250 mg, between about 500 mg and about 2000 mg, between about 500 mg and about 1500 mg, between about 500 mg and about 1200 mg, between about 500 mg and about 1000 mg, between about 750 mg and about 5000 mg, between about 1000 mg and about 5000 mg, between about 1500 mg and about 5000 mg, between about 1750 mg and about 5000 mg, between about 2000 mg and about 5000 mg, between about 2100 mg and about 5000 mg, between about 2200 mg and about 5000 mg, between about 3000 mg and about 5000 mg, between about 3500 mg and about 5000 mg, between about 4000 mg and about 5000 mg, between about 1000 mg and about 3000 mg, between about 1500 mg and about 2700 mg, between about 1800 mg and about 2600 mg, between about 2000 mg and about 2400 mg, and between about 2100 mg and about 2300 mg) of fish oil is administered. In some embodiments, a composition provided herein can be formulated to contain an amount of fish oil such that a daily dose of between about 1000 mg and about 3000 mg (e.g., between about 1500 mg and about 2700 mg, between about 1800 mg and about 2600 mg, between about 2000 mg and about 2400 mg, and between about 2100 mg and about 2300 mg) is administered. For example, a composition provided herein can be formulated such that a daily dose of about 2200 mg of fish oil is administered.

### Formulations of a Dietary Supplement

As described above, a composition provided herein can include one or more of Coenzyme Q10, magnesium, riboflavin, pine bark extract, and fish oil (e.g., Coenzyme Q10, magnesium, and riboflavin; Coenzyme Q10, riboflavin, and pine bark extract; Coenzyme Q10, pine bark extract, and fish oil; magnesium, riboflavin, and pine bark extract; magnesium, pine bark extract, and fish oil; Coenzyme Q10, riboflavin, pine bark extract, and fish oil; Coenzyme Q10, riboflavin, and fish oil; riboflavin, pine bark extract, and fish oil; and Coenzyme Q10 and pine bark extract). Such compositions can be used to prevent and/or relieve one or more symptoms of a migraine. For example, the compositions provided herein can be used to reduce the frequency of headache, the severity of headache, the duration of headache, and/or the degree of migraine disability. Additional symptoms of migraines include headache, nausea, vomiting, photophobia, phonophobia, and aura (e.g., visual, somatosensory, and motor symptoms). In some cases, the document provides methods for preventing or treating a migraine, for example, preventing and/or reducing one or more symptoms associated with a migraine. Such methods can include administering a composition provided herein.

The compositions provided herein can be ingested (e.g., orally or intragastrically), or can be administered to a mammal by other routes. For example, a composition provided herein can be administered nasally, intravenously, intramuscularly, subcutaneously, sublingually, intrathecally, or intradermally. The route of administration can depend on a variety of factors, such as the environment (e.g., the circumstances resulting in the condition or symptoms) and therapeutic goals.

When administered orally, the composition can be in the form of a tablet, soft gel, or powder. Tablets, soft gels, and powders can be configured to have a unit dosage equal to the daily desired dosage. For example, if a mammal desires 1000 mg of a particular composition, each unit dosage (e.g., soft gel) can be 1000 mg in weight. In some embodiments, tablets, soft gels, and powders can be configured to have multiple unit dosages equal to the daily desired dosage. For example, if a mammal desires 1000 mg of a particular composition, each unit dosage (e.g., each soft gel) can be 200 mg in weight and the mammal can take four unit dosages over the course of the day (e.g., two soft gels twice daily). As used herein, mammals generally refer to humans, but also can include domesticated mammals (e.g., dogs, cats, and livestock such as cows, horses, pigs, or sheep) in which preventing or treating migraine (e.g., reducing one or more symptoms associated with a migraine) is desirable.

The dosages of a particular composition will depend on many factors including the mode of administration. A dietary supplement provided herein can be formulated in a dose such that an individual receives about 37.5 mg Coenzyme Q10, about 100 mg magnesium (e.g., Magnesium Oligo), about 100 mg riboflavin, about 12.5 mg pine bark extract, and about 550 mg fish oil in a single soft gel capsule.

By way of example, a composition provided herein can be in the form of a liquid, solution, suspension, tablet, powder, cream, mist, atomized vapor, aerosol, soft gelatin capsules, or hard gelatin capsules. Commercial dietary supplements are generally formulated for oral administration. For oral administration, tablets or capsules can be prepared with pharmaceutically acceptable excipients such as binding agents, fillers, lubricants, disintegrants, or wetting agents. In some cases, the tablets can be coated. Liquid preparations for oral administration can take the form of, for example, solutions, syrups, or suspension, or they can be presented as a dry product for constitution with saline or other suitable liquid vehicle before use. Liquid preparations also can contain pharmaceutically acceptable additives such as suspending agents, emulsifying agents, non-aqueous vehicles, preservatives, buffer salts, flavoring agents, coloring agents, and sweetening agents as appropriate. Preparations for oral administration can be suitably formulated to give controlled release of one or more of the compounds. In some cases, a composition provided herein can be formulated as a soft gel capsule.

A composition provided herein can contain a pharmaceutically acceptable carrier for in vivo administration to a mammal Such pharmaceutically acceptable carriers include, without limitation, vegetable oils, glycerin, sorbitan oleate, tirethyl citrate, beeswax, and lecithin oil. In some embodiments, the carriers can include beeswax and lecithin oil.

For example, in some cases, a soft gel capsule including a composition provided herein can further include between about 0.5% and 4% by weight beeswax (e.g., between about 1% and 3.5% by weight; between about 1.5% and 3% by weight; or between about 2% and 3% by weight). In some embodiments, the beeswax is present in an amount of about 2.59% by weight. In some cases, the beeswax in the composition is between about 10 mg and about 70 mg (e.g., between about 15 mg and about 60 mg; between about 20 mg and 50 mg; between about 25 mg and about 45 mg; or between about 30 mg and 40 mg). For example, about 35 mg of beeswax can be used in the composition. In certain cases, a soft gel capsule including a composition provided herein can further include between about 0.1 and 2% by weight lecithin oil (e.g., between about 0.25% and 1.5% by weight; between about 0.5% and 1% by weight; or between about 0.6% and 0.8% by weight). For example, lecithin oil can be present in an amount of about 0.7% by weight. In some cases, lecithin oil in a composition provided herein can be between about 1 mg and about 20 mg (e.g., about 5 mg and about 15 mg; about 7.5 mg and 12.5 mg; or about 8 mg and 12 mg). For example, a composition provided herein can include about 10 mg of lecithin oil.

The compositions provided herein may be administered at once, or may be divided into a number of smaller doses to be administered at intervals of time. It is understood that for any particular subject, specific dosage regimens can be adjusted over time according to the individual need of the subject and the professional judgment of the person administering or supervising the administration of the compositions. In some embodiments, a composition provided herein can be administered at least once daily for at least 28 consecutive days (e.g., at least 56 consecutive days, at least 84 consecutive days, or at least 112 consecutive days). In some embodiments, a composition provided herein can be administered at least twice daily for at least 28 consecutive days (e.g., at least 56 consecutive days, at least 84 consecutive days, or at least 112 consecutive days).

### EXAMPLES

### Example 1

### Dietary Supplement Formulation

Soft gel capsules were prepared using amounts of each component as shown in Table 1. Amounts are given per soft gel capsule with a typical serving size of four soft gel capsules. The amount listed in the Active Claim column is shown to indicate the amount of each active that is claimed on the label. Formula amounts may be larger than active ingredient claim amounts both because the components do not necessarily include solely the active ingredient and to provide a slight overage to ensure that the finished product contains sufficient active ingredients to satisfy label claims over the life of the product.

TABLE 1

| | Active | Formula | % by |
|---|---|---|---|
| Component | Claim (mg) | amount (mg) | weight |
| Coenzyme Q10 | 37.5 | 40.94 | 3.02 |
| Magnesium (as | 100 | 586.21 | 43.30 |
| Magnesium Amino | | | |
| Acid Oligofructose | | | |
| Complex) | | | |
| Riboflavin (B2) | 100 | 117.35 | 8.67 |
| Proanthocyanadines | 12.5 | 14.38 | 1.06 |
| (from Pine Bark | | | |
| Extract) | | | |
| Fish Oil TG 18/12 | 550 | 550 | 40.62 |
| Beeswax | 35 | 35 | 2.59 |
| Lecithin oil | 10 | 10 | 0.74 |

### Example 2

### Effect of Supplement in Reduction or Severity of Migraine

A. General Protocol

1. Objective:

To assess the efficacy of the dietary supplement of Example 1 in subjects diagnosed with episodic migraine (having two to eight migraine episodes per month and its impact on: the frequency of headache; severity of headache; duration of headache; medication intake variation; migraine disability; migraine-specific quality of life; and subject's global impression of change.

2. Purpose:

The purpose of the study was to determine the effect of supplementation in reduction of migraine on human volunteers.

3. Study Design

A triple blinded, randomized, placebo controlled, 112 days study was performed wherein subjects were provided a twice daily dose (2×2 soft gel capsules) of the dietary supplement of Example 1 was conducted as follows:

A total of 100 subjects were included to participate in this study to complete 80 cases.

A total of 100 subjects who satisfy the inclusion criteria as judged by their case histories were included in this study. For each subject, the study concluded after 112 days from enrolment and included 5 follow up visits (Day 0, 28, 56, 84 and 112). After consenting to participate, subjects were put on a 28 day baseline observation period; they were randomized before being administered the test supplement or placebo.

4. Method Description:

Day 0 (A 28 day observation period with baseline daily diary entries will start)

Medical history and physical examination were performed.

Subjects with 2-8 episodes of migraine per month, with an interval of 48 hours between attacks, with or without aura and with a maximum frequency of 15 migraine days per month were selected for to participate in this study.

All subjects were asked to complete a Migraine Disability Assessments (HIT-6 or similar), a Migraine-Specific Quality of Life Questionnaire (Domains: Role Function Restrictive and Preventive and Emotional Function), the Subject's Global Impression of Change Questionnaires, and VAS Migraine Questionnaires for the following symptoms: premonitory symptoms (fatigue, concentration impairment, mood change); frequency, severity and duration of their headaches; changes in nausea and/or vomiting; and changes in photophobia, phonophobia, osmophobia. See (Dodick D, et al. Headache, 47(10): 1398-1408 (2007)).

5. Subject Selection:

A total of 300 subjects were prescreened to complete 80 cases.

Only subjects previously diagnosed with episodic migraine, with a history of no less than one year, meeting the criteria of the International Headache Society (Diagnostic Criteria for Migraine) and not currently taking a daily migraine preventative medication or currently suffering from medication overuse headache were included in the study.

ICHD-II Diagnostic Criteria for Migraine.

6. Data Analysis:

All data was analyzed via the significance of the supplements effect and was calculated using Paired-Sample Comparison Analysis (Paired T-test).

7. Adverse Events:

All adverse events were recorded and interpreted in the adverse case report form in the appropriate section by the Investigator. In addition to any other adverse reaction, events such as gastric distress, rash, or headache were considered an adverse reaction and were recorded.

B. Results

1. Overview Study protocols were reviewed by an independent Neurologist prior to study initiation. Subjects were placed into two groups: Group 1=Test Group; Group 2=Placebo Group. From the total of 80 participants, 86% were woman and 14% were male with a mean (±SD) age of 36.9±12.2, and the composition of the groups was similar between the test and placebo groups.

Significant improvements were found in frequency, severity, and duration of migraines in the test group. No significant difference was found in medication consumption.

Adverse effects, while common, were mild and of short effect. 43 adverse events occurred during the study, 36 in Group 1, and 7 in Group 2 with patients reporting mild adverse events such as constipation, stomach discomfort, soft stool, and diarrhea. Adverse events lasted between 1-3 days. No individuals discontinued use of product during the course of the study.

2. Significant Results

A. Headache Frequency (see FIGS. 1 and 2)

On day 56, Group 1 demonstrated a statistically significant reduction in headache frequency with a reduction of 26.5% with respect to baseline (Day 28) and a difference of −24.5% with respect to Group 2. On day 112, Group 1 demonstrated a statistically significant reduction in headache frequency with a reduction of 22.6% with respect to baseline (Day 28) and a difference of −25.8% with respect to Group 2. Data was reviewed by responder versus non responder, as well as those that had a greater than 40% improvement. In FIG. 2, numbers represent the percentage of individuals that had a greater than 40% improvement.

B. Headache Severity (see FIGS. 3 and 4)

On day 56, Group 1 demonstrated a statistically significant reduction in headache severity with a reduction of 33.3% with respect to baseline (Day 28). On day 84, Group 1 demonstrated a statistically significant reduction in headache severity with a reduction of 26.2% with respect to baseline (Day 28). On day 112, Group 1 demonstrated a statistically significant reduction in headache severity with a reduction of 28.8% with respect to baseline (Day 28). Data was reviewed by responder versus non responder, as well as those that had a greater than 40% improvement. In FIG. 4, numbers represent the percentage of individuals that had greater than 40% improvement.

C. Headache Duration (see FIGS. 5 and 6)

On day 112, Group 1 demonstrated a statistically significant reduction in the duration of the headaches with a reduction of 31.1% with respect to baseline (Day 28) and a difference of −23.4% with respect to Group 2. Data was reviewed by responder versus non responder, as well as those that had a greater than 40% improvement. In FIG. 6, numbers represent the percentage of individuals that had greater than 40% improvement.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A dietary supplement comprising Coenzyme Q10, magnesium, and riboflavin, wherein the improvement comprises:
pine bark extract and fish oil in combination with said Coenzyme Q10, magnesium, and riboflavin.

2. The dietary supplement of claim 1, wherein the supplement further comprises one or more of beeswax and lecithin oil.

3. The dietary supplement of claim 1, wherein the magnesium is a magnesium salt or a complex containing magnesium.

4. The dietary supplement of claim 3, wherein the complex containing magnesium is a magnesium amino acid polysaccharide complex.

5. The dietary supplement of claim 1, comprising:
(a) from about 1% to about 4% by weight Coenzyme Q10;
(b) from about 20% to about 60% by weight magnesium;
(c) from about 20% to about 60% by weight riboflavin;
(d) from about 0.25% to about 2% by weight pine bark extract; and
(e) from about 20% to about 60% by weight fish oil.

6. The dietary supplement of claim 5, comprising:
(a) from about 2.75% to about 3.25% by weight Coenzyme Q10;
(b) from about 42.5% to about 47.5% by weight magnesium;
(c) from about 42.5% to about 47.5% by weight riboflavin;
(d) from about 0.8% to about 1.2% by weight pine bark extract; and
(e) from about 38% to about 42% by weight fish oil.

7. The dietary supplement of claim 1, comprising:
(a) from about 15 mg to about 45 mg of Coenzyme Q10;
(b) from about 50 mg to about 150 mg magnesium;
(c) from about 50 mg to about 150 mg riboflavin;
(d) from about 6 mg to about 18 mg pine bark extract; and
(e) from about 200 mg to about 800 mg fish oil.

8. The dietary supplement of claim 7, comprising:
(a) about 37.5 mg of Coenzyme Q10;
(b) about 100 mg magnesium;
(c) about 100 mg riboflavin;
(d) about 12.5 mg pine bark extract; and
(e) about 550 mg fish oil.

9. A method for preventing or treating a migraine in a mammal, the method comprising administering to the mammal a therapeutically effective amount of a dietary supplement comprising Coenzyme Q10, magnesium, and riboflavin, wherein the improvement comprises:
pine bark extract and fish oil in combination with said Coenzyme Q10, magnesium, and riboflavin.

10. The method of claim 1, wherein said administering occurs at least once daily for at least 28 consecutive days.

11. A method for reducing one or more symptoms associated with a migraine in a mammal, the method comprising administering to the mammal a therapeutically effective amount of a dietary supplement comprising Coenzyme Q10, magnesium, and riboflavin, wherein the improvement comprises:
pine bark extract and fish oil in combination with said Coenzyme Q10, magnesium, and riboflavin.

12. The method of claim 11, wherein the one or more symptoms associated with a migraine are selected from the group consisting of: the frequency of headache, the severity of headache, the duration of headache, and the degree of migraine disability.

13. The method of claim 11, wherein the one or more symptoms associated with a migraine are selected from the group consisting of: headache, nausea, vomiting, photophobia, phonophobia, and aura.

14. The method of claim 9, wherein a daily dosage of the dietary supplement comprises:
(a) from about 100 mg to about 300 mg Coenzyme Q10;
(b) from about 200 mg to about 600 mg magnesium;
(c) from about 200 mg to about 600 mg riboflavin;
(d) from about 20 mg to about 80 mg pine bark extract; and
(e) from about 1000 mg to about 3000 mg fish oil.

15. The method of claim 11, wherein a daily dosage of the dietary supplement comprises:
(a) from about 100 mg to about 300 mg Coenzyme Q10;
(b) from about 200 mg to about 600 mg magnesium;
(c) from about 200 mg to about 600 mg riboflavin;
(d) from about 20 mg to about 80 mg pine bark extract; and
(e) from about 1000 mg to about 3000 mg fish oil.

16. A method for preventing or treating a migraine in a mammal, the method comprising administering to the mammal a therapeutically effective amount of a dietary supplement comprising Coenzyme Q10, magnesium, and riboflavin, wherein the improvement comprises:
pine bark extract and fish oil in combination with said Coenzyme Q10, magnesium, and riboflavin, and
wherein said administering occurs at least once daily for at least 28 consecutive days.

17. The method of claim 16, wherein said administering occurs at least once daily for at least 56 days.

18. The method of claim 17, wherein said administering occurs at least once daily for at least 84 days.

19. The method of claim 18, wherein said administering occurs at least once daily for at least 112 days.
